(19) United States
(12) Patent Application Publication
(10) Pub. No.: US 2014/0135269 A1
(43) Pub. Date: May 15, 2014

(54) Peptide And Protein-Based Formula For Improving The Appearance Of Skin

(71) Applicant: Peter Thomas Roth Labs, LLC, New York, NY (US)

(72) Inventor: Peter Thomas Roth, New York, NY (US)

(21) Appl. No.: 14/038,047

(22) Filed: Sep. 26, 2013

Related U.S. Application Data
(60) Provisional application No. 61/708,406, filed on Oct. 1, 2012.

(51) Int. Cl. A61K 8/64; A61Q 19/08

(52) U.S. Cl. CPC A61K 8/64; A61Q 19/08
USPC 514/18.8

(57) ABSTRACT

The present invention relates to methods and compositions for or treating human skin to reduce the visible appearance of fine lines and wrinkles as well as to enhance the skin's luminosity comprising applying an effective amount of a cosmetic composition of the invention to the skin for a period of time.

### FIELD OF THE INVENTION

The present invention relates to the field of cosmetics, particularly topical formulations for promoting the appearance of healthy skin by reducing fine lines and wrinkles.

### BACKGROUND OF THE INVENTION

The appearance of facial skin is accorded much weight in popular culture. In particular, a smooth, even-colored, wrinkle-free complexion is viewed as highly desirable by some. Accordingly, there have been efforts to develop new non-toxic formulations that can visibly improve the appearance of older skin by reducing the appearance of fine lines and wrinkles and providing a brighter, more even in skin tone.

Recently, new technologies for reducing the appearance of wrinkles and fine lines have targeted collagen production in skin cells. The loss of collagen with age is associated with some of the most recognizable age-related changes in human skin. These include loss of elasticity, the formation of lines, and sagging or wrinkling. The term collagen refers to a group of naturally occurring proteins abundant in mammalian skin and connective tissue. Skin collagen has an amino acid composition that is abundant in proline, glycine, and hydroxyproline. The most common types of collagen in human skin are Types I and III. Types I and III make up about 75% of the protein content of human skin and provide for the skin's mechanical strength and texture. The skin also contains Type IV collagen which forms a sheet called the lamina densa to which other anchoring peptides are connected. Type VII collagen forms anchoring fibrils in the dermal-epidermal junction connecting the dermis to the epidermis. Type XVII collagen is the major component of the hemidesmosomes. It plays an important role in anchoring the epidermis to the underlying layers of skin.

Various amino acids and peptides have reportedly been demonstrated to enhance collagen production in skin cells both by increasing collagen gene expression and increasing collagen protein production in treated cells. Others have been reported to increase the expression of particular extracellular matrix proteins, thereby improving the ability of collagen fibers to organize and promote the mechanical strength and texture of skin. Still other combinations or blends of amino acids reportedly promote healthy skin by aiding in moisture retention, providing nutrients, and promoting fibroblast proliferation. But there exists a need to provide a single formulation that effectively promotes the maintenance of collagen in human skin and visibly reduces fine lines and wrinkles while providing a brighter, more even skin tone.

### SUMMARY OF THE INVENTION

The present invention provides cosmetic compositions and methods for reducing the appearance of fine lines and wrinkles on human skin and providing skin that is generally smoother and more firm than untreated skin, while simultaneously enhancing the skin's luminosity. The term “luminosity” refers to the brightness (lightness) of the skin and the evenness of skin tone, e.g., the absence of age spots and other dark blemishes. Thus, skin having enhanced luminosity is skin that is noticeably healthier, brighter, and more even in tone and pigmentation compared to untreated skin.

In one embodiment, the invention provides a method for treating human skin to reduce the visible appearance of fine lines and wrinkles as well as to enhance the skin's luminosity comprising applying an effective amount of a cosmetic composition of the invention to the skin for a period of time. In one embodiment, the period of time is at least about one to five weeks. In one embodiment, the cosmetic composition is applied at least daily. In another embodiment, the cosmetic composition is applied more than once a day, for example twice or three times a day. In one embodiment, the cosmetic composition is applied once a day. In one embodiment, the human skin is facial skin.

In one embodiment, the invention provides a cosmetic composition for topical application to skin, the composition providing for the percutaneous administration of a blend of amino acids and peptides to the skin, wherein the combination of amino acids and peptides promotes the production of collagen in human cells. In one embodiment, the blend comprises two or more amino acids selected from the group consisting of lysine, histidine, arginine, aspartic acid, threonine, serine, glutamic acid, proline, glycine, alanine, valine, methionine, isoleucine, leucine, tyrosine, henylalanine, cysteine, and N-acetyl-L-hydroxyproline. In one embodiment, the blend comprises a peptide selected from lysine-valine-lysine and lysine-valine-threonine. In certain embodiments, the peptide is modified by the covalent attachment of one or moieties, for example, a fatty acid, hydroxyl, or butyrate moiety. In one embodiment the peptide is modified by palmitoylation.

In one embodiment, the cosmetic composition further comprises one or more amino acids or peptides that do not promote the production of collagen in human cells. In one embodiment, the one or more amino acids or peptides enhances the production of extracellular matrix proteins in human cells. In one embodiment, the cosmetic composition further comprises one or more amino acids or peptides that inhibits the production of melanin in human cells. In another embodiment, the cosmetic composition further comprises a blend of amino acids that enhance moisture retention of skin and promote the proliferation of skin fibroblast cells.

### DETAILED DESCRIPTION OF THE INVENTION

The cosmetic compositions and methods of the present invention advantageously promote the maintenance of collagen in human skin thereby minimizing the age-related changes that are incident to the loss of collagen including, but not limited to, the loss of elasticity, the formation of lines, sagging, and wrinkling. The present invention also provides methods for promoting the healthy appearance of skin by enhancing the skin's luminosity.

The methods of the present invention comprise methods for treating skin comprising applying to the surface of the skin an effective amount of a cosmetic composition of the invention. Preferably, the method comprises applying the cosmetic composition to the surface of the skin for a period of time that is at least one to three days, preferably at least seven days. In certain embodiments, the method comprises applying the composition for a period of time that is from one to five weeks, or from two to six weeks, or from three to six weeks. In one embodiment, the composition is applied for a period of time that is at least one week, at least two weeks, at least three weeks, at least four weeks or at least five weeks.

In accordance with the methods of the invention, the treated skin has demonstrably improved properties compared to skin that has not been treated or untreated skin. In certain embodiments, the properties are selected from the group consisting of elasticity, resistance to oxidative damage, smoothness, plumpness, moisture retention, visible wrinkles, visible lines, firmness, and skin tone. In certain embodiments, visible wrinkles are measured by one or more of a combination of parameters including wrinkle depth, wrinkle area, wrinkle volume.

The present invention also provides cosmetic compositions for achieving a smoother, healthier appearance of human skin by reducing the visible fine lines and wrinkles and by enhancing the skin's luminosity. The term “luminosity” refers to the brightness (lightness) of the skin and the evenness of skin tone, e.g., the absence of age spots and other dark blemishes. Thus, skin having enhanced luminosity is skin that is noticeably healthier, brighter, and more even in tone and pigmentation compared to untreated skin.

The cosmetic compositions of the invention provide for the percutaneous administration of a blend of two or more biologically active agents to the skin. The term biologically active in this context means the ability to induce a cellular response, for example to induce or increase cell proliferation, or to increase or decrease the expression of a gene or a protein in a living cell. In one embodiment, the cellular response is increased production of collagen protein. Preferably, the two or more biologically active agents are amino acids or peptides. The peptides preferably consist of three to five amino acids. In one embodiment, a peptide for use in the compositions of the invention consists of three, four, or five amino acids. In another embodiment, a a peptide for use in the compositions of the invention consists of three to six amino acids or five to ten amino acids. The biologically active amino acids or peptides preferably are active in promoting the production of collagen in human cells. In some embodiments, the biologically active amino acids or peptides are active in promoting the production of one or more extracellular matrix proteins in human cells, for example fibroblast cells.

In one embodiment, the blend of two or more biologically active agents comprises two or more amino acids selected from the group consisting of lysine, histidine, arginine, aspartic acid, threonine, serine, glutamic acid, proline, glycine, alanine, valine, methionine, isoleucine, leucine, tyrosine, henylalanine, cysteine, and N-acetyl-L-hydroxyproline. In one embodiment, the blend comprises a peptide selected from lysine-valine-lysine and lysine-valine-threonine. In certain embodiments, the peptide is modified by the covalent attachment of one or moieties, for example, a fatty acid, hydroxyl, or butyrate moiety. In one embodiment the peptide is modified by palmitoylation. Amino acids and peptides suitable for use in the present invention are commercially available, for example, from Bioland (SongJeong, Korea) and under tradenames such as SYN-TC (Pentapharm, Basal, Switzerland), AHYP (Kyowa Hakko Bio Co. Ltd.), DERMICAN LS (Laboratoires Serobiologiques, Pulnoy, France) and DERMOSTATYL IS (Ashland, Inc, Covington, Ky.).

Other peptides for use in the cosmetic compositions of the invention include certain di-, tri-, tetra-, penta- and hexapeptides selected from the group consisting of carnosine (beta-alanyl-L-histidine), Gly-His-Lys, Arg-Lys-Arg, and His-Gly-Gly, palmitoyl-Gly-His-Lys, Arg-Ser-Arg-Lys, palmitoyl-Lys-Thr-Thr-Lys-Ser, and Glu-Glu-Met-Gln-Arg-Arg.

In one embodiment, the cosmetic compositions of the invention further comprise luminosity enhancer such as mica.

In one embodiment, the cosmetic compositions of the invention further comprise one or more soluble collagens. Soluble collagens suitable for use in the formulations of the invention include those available under the tradenames COLLASOL M (Croda, Intl. PLC), COLLAGENON (Vevy Europe), and ACACIA COLLAGEN (Cosmetochem Intl. AG).

The cosmetic compositions of the invention preferably also provide enhanced moisturization and moisture retention to treated areas of the skin. In one embodiment, the compositions comprise a moisturizing component comprising one or more ingredients selected from the group consisting of water, sodium hyaluronate, glycerin, algae extract, soy extract, yeast extract, glutamine, glutamic acid, and sodium hyaluronate.

Moisturizers suitable for use in the formulations of the invention include those available under the tradenames HYASOL (sodium hyaluronate) and DERMALRX SRC (Biocogent LLC). In certain embodiments, the compositions of the invention further provide enhanced luminosity to the treated skin. In one embodiment, the compositions of the invention farther provide protection against oxidative damage to the skin.

The cosmetic compositions of the invention may take any form suitable for topical application to the skin. For example, the compositions may be in the form of emulsions, milks, lotions, ointments, pomades, shampoos, soaps, gels, powders, body oils, face masks and plasters.

The cosmetic compositions of the invention may further comprise one or more other commonly used ingredients of topical cosmetic preparations including lipids, gel-forming and viscous polymers, surfactant and emulsifying polymers, botanical extracts, and sunscreens. For example, the compositions of the invention may comprise one or more of the following additional excipients: Acetyl Glucosamine, Ascorbate, Alcohol, Allantoin, Ammonium Hydroxide, Avena Sativa (Oat) Kernel Extract, Butylene Glycol, Butylparaben, Caprylic/Capric Triglyceride, Chlorella Vulgaris Extract, Cyathea Cumingli Leaf Extract, Disodium EDTA, Ethylparaben, Glycerin, Glyceryl Acrylate/Acrylic Acid Copolymer, Hydrogenated Polyisobutene, Helianthus Annuus (Sunflower) Seed Oil, Hydroxyethylcellulose, Isobutylparaben, Lavandula Angustifolia (Lavender) Oil, Lecithin, Magnesium Ascorbyl Phosphate, Methylparaben, Panthenol, Phenoxyethanol, Polysorbate 20, Polysorbate 80, Propylene Glycol, Silica Dimethyl Silylate, Sodium Bisulfite, Phospholipids, Propylparaben, Polyglyceryl-10 Stearate, Sorbitol, Titanium Dioxide, Tocopheryl Acetate, Trimethicone, Triethanolamine, Salvia Selarea (Clary) Oil, Xanthan Gum,

In certain embodiments, the compositions comprise one or more additional excipients selected from the group consisting of crosslinked polyacrylate polymer, sodium hydroxide, PEG-40 hydrogenated castor oil, trideceth-9, propylene glycol, isopropyl myristate, sodium PCA, pentylene glycal, polymethylsilsesquioxarie, PEG-10 dimethicone, sodium benzoate, potassium sorbate, and phenoxyethanol.

### Equivalents

Those skilled in the art will recognize or be able to ascertain using no more than routine experimentation, many equivalents to the specific embodiments of the invention described herein. Such equivalents are intended to be encompassed by the following claims.

All references cited herein are incorporated herein by reference in their entirety and for all purposes to the same extent as if each individual publication or patent or patent application was specifically and individually indicated to be incorporated by reference in its entirety for all purposes.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

What is claimed:

1. A method for treating human skin to reduce the visible appearance of fine lines and wrinkles as well as to enhance the skin's luminosity comprising applying an effective amount of a cosmetic composition of the invention to the skin for a period of time.

2. A cosmetic composition comprising a blend of biologically active amino acids and peptides, wherein at least two of the biologically active amino acids or peptides has the activity of increasing collagen production in human cells.
